(19) United States
(12) Patent Application Publication
(10) Pub. No.: US 2006/0224208 A1
(43) Pub. Date: Oct. 5, 2006

(54) MEDICAL ELECTRONICS ELECTRICAL IMPLANTABLE MEDICAL DEVICES

(75) Inventor: Jacques Naviaux, Rancho Palos Verdes, CA (US)

Correspondence Address: Walter A. Hackler, Suite B, 2372 S.E. Bristol Street, Newport Beach, CA, 92660-0755, US

(73) Assignee: Bal Seal Engineering Co., Inc.

(21) Appl. No.: 11/278,450

(22) Filed: Apr. 3, 2006

Related U.S. Application Data
(60) Provisional application No. 60/668,483, filed on Apr. 5, 2005.

(51) Int. Cl. A61N 1/375

(52) U.S. Cl. 607/37

(57) ABSTRACT

An implantable medical device includes an impulse generator having a header and a contact assembly which includes at least one molded plastic housing having two halves joined together with the halves defining a bore therethrough. A canted coil spring is disposed within the housing halves along a bore circumference and includes a weld plate which extends to an exterior of the housing halves. A weld plate overlays the housing halves exterior which is joined to both the spring lead and devices wires.

The present application claims priority from U.S. Provisional Application Ser. No. 60/668,483 filed Apr. 5, 2005 which is incorporated in its entirety into the present application.

This invention generally relates to implantable medical devices and is more particularly directed to a pulse generator an implantable lead or leads and a conductive path therebetween.

Implantable medical electronics devices typically consist of an implanted pulse generator for providing electrical stimulation to targeted tissues and an implantable lead or leads that are used to transmit electrical impulse to the targeted tissues. Early devices were developed for cardiac pacemaking, and now such devices have a number of applications for cardiac rhythm management, treatment for congestive heart failure, and implanted defibrillators. Other devices are used for neuromodulation with a wide range of uses such as pain control, nervous tremor mitigation, incontinence treatment, epilepsy seizure reduction, vagus nerve stimulation for clinical depression and others. This rapidly growing field will undoubtedly have even wider application in the future.

In general, such devices include an implanted impulse generator that may also be capable of sensing body activity such as an irregular heart-beat. The impulse generator may generate an electrical impulse or signal that is transmitted to a targeted tissue or tissues or nerve area or areas through an implanted lead. Once the lead or leads are implanted in the body, removal may involve major surgery with an attendant risk factor. Therefore, a reliable method of connecting and disconnecting the leads is required since the implanted impulse generator may have to be replaced to update the unit or to replace the battery.

These devices are hermetically sealed and include circuitry and a power supply. Current practice is to place a molded header containing a connector on top of the unit to provide a means of housing the electrical contacts for the leads. While some applications are very simple requiring only two leads because they only have to transmit two discrete signals to the tissues, others are very complex and require a very large number of discrete electrical impulses. Each electrical impulse requires a discrete conductive path between the impulse generator and the implanted lead.

Several different types of contacts are now in use ranging from setscrews to various types of spring contacts. These contacts are embedded in the connector which is generally made of a silicon filled implantable polymeric. The lead generally consists of a series of conductive rings separated by insulative spaces so that when it is fully inserted into the header, each conductive ring is placed in contact with a connector contact. Each contact in turn has to be connected to a discrete lead from the impulse generator.

In current practice, the connector generally consists of a setscrew in a metal connector or some type of spring in a metal housing. Where the spring is used, it provides the conductive path between the metal housing and the conductive ring of the lead. Setscrews are very undesirable where large numbers of connectors are required because each contact must be tightened with a torque wrench. A spring retained in a metal housing provides a reliable contact with controlled insertion forces that is convenient for both insertion and removal and obviates the requirement for a torque wrench. A canted coil spring has very desirable characteristics for this application since its nearly constant force over a wide range of deflection compensates for any irregularities on the surfaces of the lead electrical contact rings and the insertion force can be controlled.

The housings, which can number anywhere from two to twenty-four or even more are now machined from metals such as MP35N, titanium, or even platinum, are significant cost drivers. Therefore, it is highly desirable to utilize an implantable polymeric biocompatible material for the housing that can be fabricated by injection molding to reduce the cost of the contacts. However, an electrical path must be added to the plastic housing.

The present invention utilizes a spring with a spring lead for providing the electric path which is connected to a lead from the pulse generator. The housing is molded from an implantable polymeric material in two pieces with the spring lead extending from the housing. Various techniques may be used to attach the spring lead to the lead from the impulse generator. This connection method minimizes contact resistance and provides for a very robust electrical contact by using a weld plate as an electrical bus.

### SUMMARY OF THE INVENTION

An implantable medical device in accordance with the present invention generally includes an impulse generator having a header along with at least one contact assembly disposed within the header. The contact assembly in turn includes at least one molded plastic housing including two halves joinable together with the joined halves defining a bore therethrough. The use of molded plastic for the housing enables fabrication by injection molding which significantly reduces the cost of the assembly.

A canted coil spring is disposed within the housing halves along a bore circumference. A spring lead is welded to the canted coil spring and extends to an exterior of the housing halves.

A weld plate is provided which overlays the housing halves exterior and is joined at the spring lead. In addition, at least one device wire is also joined to the weld plate.

The assembly may include a plurality of molding plastic housings engaged with one another and aligned with coaxial bores with each of the plurality including a canted coil spring with a spring lead extending to an exterior of a corresponding housing and joined to a corresponding weld plate.

### BRIEF DESCRIPTION OF THE DRAWINGS

The advantages and features of the present invention will be better understood by the following description when considered in conjunction with the accompanying drawings, in which:

FIG. 1 is a plan view of a generator illustrating a header which includes a plurality of molded plastic housings each having joined halves for defining a bore therethrough along with an implantable lead;

FIG. 2 is a plan view of one of the implantable leads shown in FIG. 1 illustrating spaced apart conductive rings;

FIG. 3 is a an exploded perspective view of housing pairs joined with an aligned bore therethrough along with a canted coil spring disposed within the housing bores and a weld plate;

FIG. 4 is a side view of a housing halve with the canted coil spring disposed therein more clearly illustrating attachment of the weld plate to the canted coil spring and a device wire joined to the weld plate; and

FIG. 5 is an enlarged view of the weld plate and housing halves with dovetails for receiving the weld plate.

### DETAILED DESCRIPTION

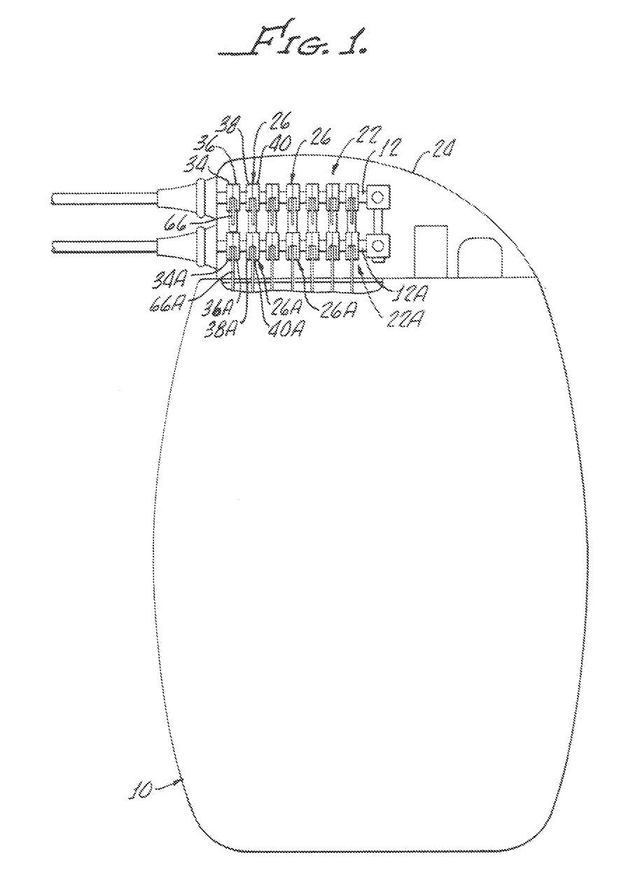

With reference to FIG. 1, there is shown an impulse generator 10 for generating electrical pulses that deliver to target tissue (not shown) by implantable leads 12, 12A.

As illustrated in FIG. 2, the implanted lead 12 includes a plurality of spaced apart conductive rings 14 separated by insulators 18, the lead 12A, not separately shown, has identical structure.

It should be appreciated that the generator 10 may also be a receptor and processor of information from the target tissues through the implanted leads 12, 12A.

As hereinabove briefly noted, the generator 10 includes a wide range of uses such as cardiac rhythm management, implanted defibrillators, and neurostimulators used for the control of pain, treatment of nervous disorders, incontinence, clinical depression, and other applications. However, the fundamental requirements are generally the same for all of these applications. That is, an electrical signal must be transmitted from the generator 10 to the leads 12, 12A and thereafter to the target tissue. Conversely, the generator 10 may, in fact, act as a receiver for gathering information from the targeted tissue through the implanted leads 12, 12A.

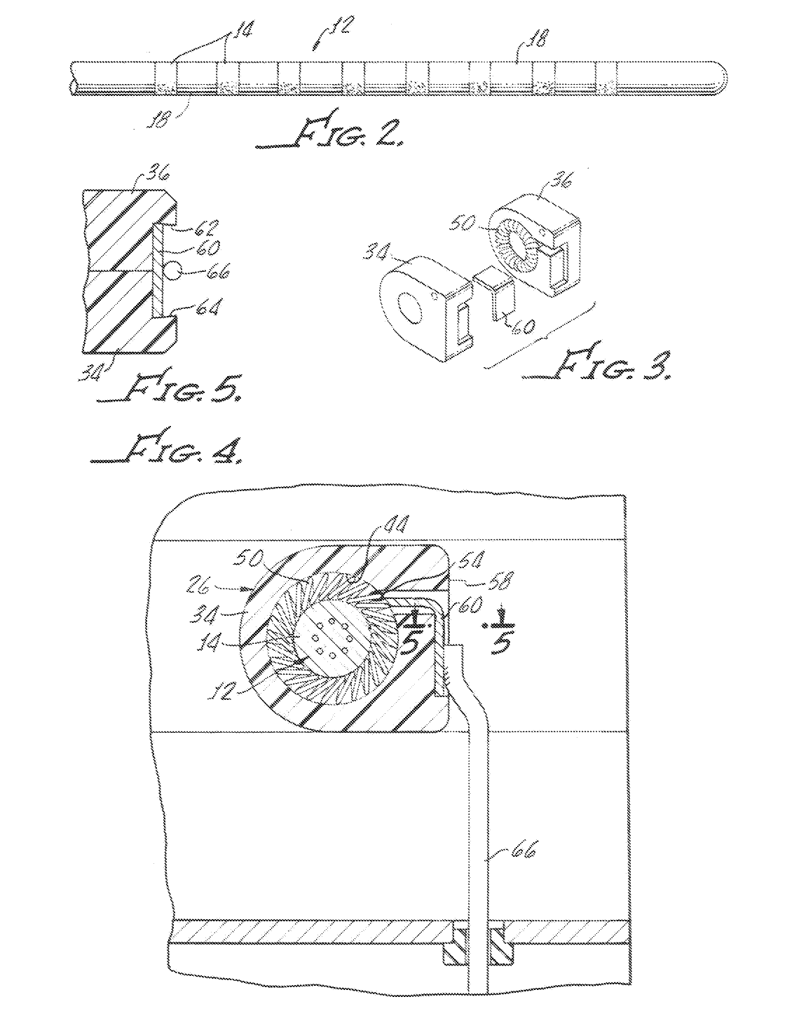

As illustrated in FIGS. 1, 3, and 4 an electrical path is provided by weld plate contact assemblies 22, 22A disposed in a header 24 (FIG. 1) which includes molded housings 26, 26A, 28, 28A formed from a polymeric biocompatible material suitable for fabrication by injection molding.

The housings 26, 26A, 28, 28A include housing halves 34, 34A, 36, 36A, 38, 38A, 40, 40A respectively which are adjoined together to form a bore 44 therethrough, see FIG. 4, only one bore 44 being shown for clarity. A canted coil spring 50 is disposed within the housing halves 34, 36 and joined, preferably by a weld 54, to a weld plate 60 which overlays the housing halves exterior 58, again only one spring 50 being shown. The housing may be joined in any manner such as by gluing or ultrasonic welding and dovetails 62, 64, see FIG. 5.

At least one device wire 66 is also joined to the weld plate 60 for providing electrical continuity between the impulse generator 10 and a conductive ring through the spring 50, spring lead 54, and weld plate 60.

As illustrated, the housings 26 with the housing halves 34, 36, 38, 40 are aligned with one another to define the bore 44 into which the implantable lead 12 is inserted.

It should be appreciated that the present invention is directed to an implantable medical device and contact assembly which is modular in concept and accordingly any number of electrical contacts may be provided. The lead 66 from the impulse generator 10 transmits electrical impulse from the generator 10 to the implanted leads 12, 12A through the contact assembly 22, 22A. As shown there is multiplicity of contacts with each contact carrying a discrete signal. The number of contacts can vary from two to twenty-four or higher for each lead 12, 12A and the electrical pulse travels from the impulse generator 10 through the contact assemblies 22, 22A through the weld plate 60 and springs 50 to the contact rings 14.

The weld plate 60 provides for a robust electrical connection despite the use of a polymeric housings 26, 28.

Although there has been hereinabove described a specific implantable medical device in accordance with the present invention for the purpose of illustrating the manner in which the invention may be used to advantage, it should be appreciated that the invention is not limited thereto. That is, the present invention may suitably comprise, consist of, or consist essentially of the recited elements. Further, the invention illustratively disclosed herein suitably may be practiced in the absence of any element which is not specifically disclosed herein. Accordingly, any and all modifications, variations or equivalent arrangements which may occur to those skilled in the art, should be considered to be within the scope of the present invention as defined in the appended claims.

What is claimed is:

1. An implantable medical device comprising:
an impulse generator having a header; and
at least one contact assembly disposed within said header and including:
at least one molded plastic housing including two halves joined together, the joined halves defining a bore therethrough;
a canted coil spring disposed within the housing halves along a bore circumference;
an exterior weld plate overlaying and aligning the housing halves and joined to the spring; and
at least one device wire joined to said weld plate.

2. The assembly according to claim 1 further comprising a plurality of molded plastic housings engaged with one another and aligned with coaxially bores each of the plurality including a canted coil spring with a weld plate extending to an exterior of the corresponding housing and joined thereto.

3. The assembly according to claim 2 further comprising a plurality of device wires, each device wire joined to a corresponding weld plate.

4. The assembly according to claim 3 further comprising an implantable lead insertable into the coaxial bores and having spaced apart conductive rings alignable with corresponding canted coil springs.

5. The assembly according to claim 1 wherein said weld plate is fixed to the housing halves by dovetails formed therein.

6. An implantable medical device comprising:
an impulse generator having a header; and
at least two parallel contact assemblies disposed within said header each assembly including:
a plurality of molded plastic housings each having including two halves joined together, the joined halves define a bore therethrough;
a canted coil spring disposed within each of the housing halves along a bore circumference;
an exterior weld plate overlaying and aligning pairs of the housing halves and joined to the spring; and
at least one device wire joined to each weld plate.

7. The assembly according to claim 6 wherein the plurality of molded plastic housings are aligned with coaxially bores.

8. The assembly according to claim 7 further comprising a plurality of device wires, each device wire joined to a corresponding weld plate.

9. The assembly according to claim 8 further comprising a pair of implantable leads insertable into corresponding coaxial bores and having spaced apart conductive rings alignable with corresponding canted coil springs.

10. The assembly according to claim 6 wherein said weld plate is fixed to the housing halves by dovetails formed therein.
